(19) United States
(12) Patent Application Publication
(10) Pub. No.: US 2024/0238182 A1
(43) Pub. Date: Jul. 18, 2024

(54) OIL-IN-WATER EMULSION COSMETIC

(71) Applicant: SHISEIDO COMPANY, LTD., Tokyo (JP)

(72) Inventors: Ryota KOMAI, Tokyo (JP); Ikuhiro SUZUKI, Tokyo (JP)

(73) Assignee: SHISEIDO COMPANY, LTD., Tokyo (JP)

(21) Appl. No.: 18/686,007

(22) PCT Filed: Sep. 9, 2022
(86) PCT No.: PCT/JP2022/033853

§ 371 Date: Feb. 23, 2024

(30) Foreign Application Priority Data
Sep. 24, 2021 (JP) 2021-155682

(51) Int. Cl. A61K 8/49; A61K 8/06; A61K 8/67; A61K 8/81; A61K 8/86; A61Q 19/00

(52) U.S. Cl. CPC A61K 8/4946; A61K 8/062; A61K 8/675; A61K 8/8147; A61K 8/86; A61Q 19/00; A61K 2800/10

(57) ABSTRACT

[Problem] To provide an oil-in-water emulsion cosmetic having an excellent feel in use.

[Solution] An oil-in-water emulsion cosmetic including (A) a cyclic carboxamide derivative having a specific structure or its salt, (B) an alkyl-modified carboxyvinyl polymer, (C) an oil, and (D) water.

### TECHNICAL FIELD

The present invention relates to an oil-in-water emulsion cosmetic.

### BACKGROUND ART

In cosmetics, it is known that an alkyl-modified carboxyvinyl polymer is blended as an emulsifier and capable of imparting a fresh feel in use and suppressing a sticky feel after application. For example, combination use of an alkyl-modified carboxyvinyl polymer with niacinamide has been proposed (Patent Literature 1). The users of such a cosmetic have desired a more excellent feel in use, and there has been room for further improvement in the feel in use.

Meanwhile, a cyclic carboxamide derivative is known to have an anti-wrinkle effect and a pigmentation inhibitory effect, and is proposed to be blended in a cosmetic and the like (Patent Literature 2).

### CITATION LIST

### Patent Literature

Patent Literature 1: WO 2021/075431 A
Patent Literature 2: WO 2011/040496 A

### SUMMARY OF THE INVENTION

According to the studies of the present inventors, it has been found that use of a cyclic carboxamide derivative in a cosmetic tends to cause a sticky feel. The present inventors have surprisingly found that an excellent feel in use can be achieved by using an oil-in-water cosmetic including a combination of an alkyl-modified carboxyvinyl polymer and a specific cyclic carboxamide derivative. The present invention is based on these findings.

According to the present invention, the following invention is provided.

[1] An oil-in-water emulsion cosmetic including:
(A) a cyclic carboxamide derivative or a salt of the cyclic carboxamide derivative, the cyclic carboxamide derivative represented by Formula (1):

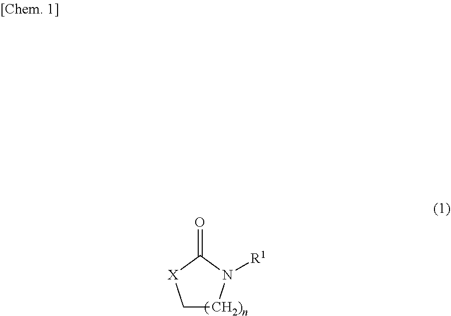

wherein
R¹ represents a hydrocarbon group having 1 to 6 carbon atoms optionally substituted with a hydroxy group or R¹ represents a hydrogen atom,
X represents —CH₂— or —N(R²)— wherein R² represents a hydrocarbon group having 1 to 6 carbon atoms optionally substituted with a hydroxy group or R² represents a hydrogen atom, and
n is an integer of 1 to 3;
(B) an alkyl-modified carboxyvinyl polymer;
(C) an oil; and
(D) water.

[2] The oil-in-water emulsion cosmetic according to [1],
wherein in Formula (1) of the component (A),
R¹ represents a hydroxyalkyl group having 1 to 3 carbon atoms,
X represents —CH₂— or —NH—, and
n is 1.

[3] The oil-in-water emulsion cosmetic according to [1] or [2], wherein the component (A) is 1-(2-hydroxyethyl)-2-imidazolidinone.

[4] The oil-in-water emulsion cosmetic according to claims [1] to [3], wherein an amount of the component (A) blended is 0.05 to 5 mass % with respect to a total amount of the oil-in-water emulsion cosmetic.

[5] The oil-in-water emulsion cosmetic according to any one of [1] to [4], wherein the component (B) is an acrylates/C10-30 alkyl acrylate crosspolymer.

[6] The oil-in-water emulsion cosmetic according to any one of [1] to [5], wherein an amount of the component (C) blended is 30 mass % or less with respect to the total amount of the oil-in-water emulsion cosmetic.

[7] The oil-in-water emulsion cosmetic according to any one of [1] to [6], further including (E) an alkylene oxide derivative represented by Formula (2):

R³O-[(AO)ₐ(EO)_{b}]—R⁴ (2)
wherein
R³ and R⁴ each independently represent an alkyl group having 1 to 4 carbon atoms,
AO represents an oxyalkylene group having 3 or 4 carbon atoms,
EO represents an oxyethylene group,
a and b represent average numbers of moles of added AO and EO, respectively, and satisfy 1≤a≤70 and 1≤b≤70, and
M_{AO} representing a molecular weight of AO and M_{EO} representing a molecular weight of EO satisfy [M_{EO}×b/(M_{AO}×a+M_{EO}×b)]=0.2 to 0.8.

[8] The oil-in-water emulsion cosmetic according to any one of [1] to [7], further including (F) nicotinic acid or a derivative of nicotinic acid.

[9] The oil-in-water emulsion cosmetic according to any one of [1] to [8], further including (G) a nonionic surfactant.

[10] The oil-in-water emulsion cosmetic according to [9], wherein an amount of the component (G) blended is 1.5 mass % or less with respect to the total amount of the oil-in-water emulsion cosmetic.

According to the present invention, an oil-in-water emulsion cosmetic having an excellent feel in use can be provided. In particular, freshness is kept during application, a sticky feel is suppressed after application, and a moist feel can be imparted to the skin.

### BEST MODE FOR CARRYING OUT THE INVENTION

The present invention relates to an oil-in-water emulsion cosmetic (hereinafter, sometimes referred to as a cosmetic) including (A) a cyclic carboxamide derivative having a specific structure or its salt, (B) an alkyl-modified carboxyvinyl polymer, (C) an oil, and (D) water.

### (A) Cyclic Carboxamide Derivative or its Salt

The cosmetic according to the present invention includes a cyclic carboxamide derivative represented by Formula (1) or its salt (hereinafter, sometimes referred to as a component (A), and the same applies to other components).

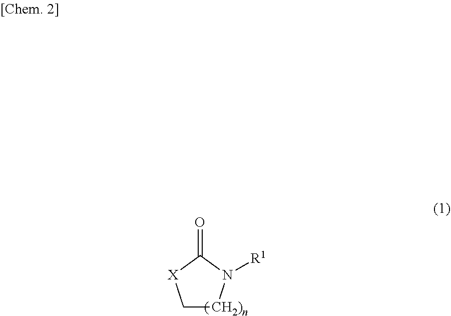

In Formula (1),
R¹ represents a hydrocarbon group having 1 to 6 carbon atoms optionally substituted with a hydroxy group or R¹ represents a hydrogen atom,
X represents —CH₂— or —N(R²)— wherein R² represents a hydrocarbon group having 1 to 6 carbon atoms optionally substituted with a hydroxy group or R² represents a hydrogen atom, and
n is an integer of 1 to 3

The hydrocarbon group is not particularly limited, and may be, for example, an alkyl group, a cycloalkyl group, an alkenyl group, an alkynyl group, a cycloalkylalkyl group, a haloalkyl group, an alkoxyalkyl group, or an alkoxycarbonylalkyl group, and is preferably an alkyl group.

In a preferred embodiment, in Formula (1) of the component (A),
R¹ represents a hydroxyalkyl group having 1 to 3 carbon atoms,
X represents —CH₂— or —NH—, and
n is 1.

Specific examples of the cyclic carboxamide derivative represented by Formula (1) include the following.

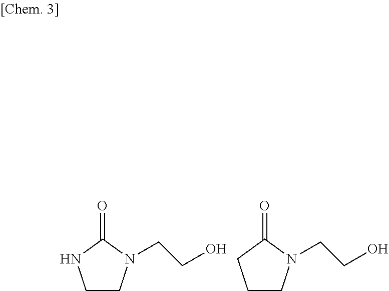

The component (A) is most preferably 1-(2-hydroxyethyl)-2-imidazolidinone.

The component (A) may be a salt of the cyclic carboxamide derivative represented by Formula (1). The kind of the salt is not particularly limited as long as the salt is pharmacologically acceptable, and the salt may be an inorganic salt or an organic salt. Examples of the inorganic salt include a hydrochloride, a sulfate, a phosphate, a hydrobromide, a sodium salt, a potassium salt, a magnesium salt, a calcium salt, a magnesium salt, and an ammonium salt. Examples of the organic salt include an acetate, a lactate, a maleate, a fumarate, a tartrate, a methanesulfonate, a p-toluenesulfonate, a triethanolamine salt, and an amino acid salt.

One or more kinds of the component (A) can be blended. The amount of the component (A) blended is preferably 0.05 to 5 mass %, more preferably 0.08 to 3.5 mass %, and still more preferably 0.5 to 2.0 mass % with respect to the total amount of the cosmetic.

### (B) Alkyl-Modified Carboxyvinyl Polymer

The cosmetic according to the present invention includes (B) an alkyl-modified carboxyvinyl polymer. The component (B) is an acrylic acid-alkyl methacrylate copolymer, and is a polymer in which at least a part of carboxyl groups of a polymer having acrylic acid or methacrylic acid as a main chain is esterified with an alkyl group. The alkyl group bonded by an ester bond may be linear or branched, and the number of carbon atoms is preferably 10 to 30. The component (B) is preferably a crosspolymer in which polymers or copolymers including alkyl acrylate or alkyl methacrylate as a polymerization unit are crosslinked with each other. Examples of the component (B) include an acrylates/C10-30 alkyl acrylate crosspolymer.

One or more kinds of the component (B) can be blended. The amount of the component (B) blended is preferably 0.005 to 1.5 mass %, and more preferably 0.01 to 0.5 mass % with respect to the total amount of the cosmetic.

### (C) Oil

The cosmetic according to the present invention includes (C) an oil. Examples of the component (C) include silicone oils, hydrocarbon oils, higher fatty acids, higher alcohols, ester oils, liquid oils, solid oils, and semi-solid oils, and a silicone oil or a hydrocarbon oil is preferable, and a silicone oil, a hydrocarbon oil, and an ester oil are more preferable.

Examples of the silicone oils include chain polysiloxanes (such as dimethicone, diphenylsiloxy phenyl trimethicone, and diphenylpolysiloxane), cyclic polysiloxanes (such as octamethylcyclotetrasiloxane, decamethylcyclopentasiloxane, and dodecamethylcyclohexasiloxane), silicone resins forming a three-dimensional network structure, silicone rubber, various modified polysiloxanes (such as an amino-modified polysiloxane, a polyether-modified polysiloxane, an alkyl-modified polysiloxane, and a fluorine-modified polysiloxane), and acrylic silicones, and chain polysiloxanes are preferable.

Examples of the hydrocarbon oils include isododecane, isohexadecane, isoparaffin, mineral oil (liquid paraffin), ozokerite, squalane, pristane, paraffin, ceresin, squalene, petrolatum, microcrystalline wax, and hydrogenated polydecene.

Examples of the higher fatty acids include lauric acid, myristic acid, palmitic acid, stearic acid, behenic acid, oleic acid, undecylenic acid, tall oil acid, isostearic acid, linolic acid, linoleic acid, eicosapentaenoic acid (EPA), and docosahexaenoic acid (DHA).

Examples of the higher alcohols include straight-chain alcohols (such as lauryl alcohol, cetyl alcohol, stearyl alcohol, behenyl alcohol, myristyl alcohol, oleyl alcohol, and cetostearyl alcohol) and branched alcohols (such as lanolin alcohol, cholesterol, phytosterol, hexyldodecanol, isostearyl alcohol, and octyldodecanol).

Examples of the ester oils include octyl octanoate, nonyl nonanoate, cetyl octanoate, isopropyl myristate, octyldodecyl myristate, isopropyl palmitate, ethylhexyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, cholesteryl 12-hydroxystearate, ethylene glycol di-2-ethylhexanoate, dipentaerythritol fatty acid ester, N-alkylglycol monoisostearate, neopentyl glycol dicaprate, tripropylene glycol pivalate, diisostearyl malate, glycerin di-2-heptylundecanoate, glycerin diisostearate, trimethylolpropane tri-2-ethylhexanoate, trimethylolpropane triisostearate, pentaerythritol tetra-2-ethylhexanoate, glycerin tri-2-ethylhexanoate, glycerin trioctanoate, glycerin triisopaImitate, trimethylolpropane triisostearate, cetyl 2-ethylhexanoate-2-ethylhexyl palmitate, glycerin trimyristate, glyceride tri-2-heptylundecanoate, castor oil fatty acid methyl ester, oleyl oleate, acetoglyceride, 2-heptylundecyl palmitate, diisobutyl adipate, N-lauroyl-L-glutamate-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, triethyl citrate, cetyl ethylhexanoate, and phytosteryl macadamiate.

Examples of the liquid oils include avocado oil, camellia oil, macadamia nut oil, corn oil, olive oil, rapeseed oil, sesame oil, persic oil, wheat germ oil, sasanqua oil, castor oil, linseed oil, safflower oil, cottonseed oil, perilla oil, soybean oil, peanut oil, tea seed oil, kaya oil, rice bran oil, paulownia oil, Japanese tung oil, jojoba oil, germ oil, and triglycerin oil. Examples of the solid oils include cacao butter, coconut oil, hardened coconut oil, palm oil, palm kernel oil, hydrogenated palm oil, Japan wax kernel oil, hardened oils, Japan wax, and hardened castor oil. Examples of the semi-solid oils include shea butter, partially hydrogenated coconut oil, and partially hydrogenated jojoba oil.

One or more kinds of the component (C) can be blended. The amount of the component (C) blended is preferably 30 mass % or less, more preferably 0.1 to 20 mass %, still more preferably 0.5 to 15 mass %, and particularly preferably 1.0 to 8.5 mass % with respect to the total amount of the cosmetic.

### (D) Water

The cosmetic according to the present invention includes (D) water. As the water, water used in cosmetics, quasi-drugs, and the like can be used, and for example, purified water, ion-exchanged water, tap water, or the like can be used.

The amount of the water blended is preferably 20 to 95 mass %, and more preferably 60 to 90 mass % with respect to the total amount of the cosmetic according to the present invention.

### (E) Alkylene Oxide Derivative

The cosmetic according to the present invention can further include (E) an alkylene oxide derivative represented by Formula (2).

R³O-[(AO)ₐ(EO)_{b}]—R⁴ (2)

In the Formula (2),
R³ and R⁴ each independently represent an alkyl group having 1 to 4 carbon atoms, and examples thereof include a methyl group, an ethyl group, an n-propyl group, an isopropyl group, an n-butyl group, a sec-butyl group, and a tert-butyl group, and a methyl group or an ethyl group is preferable.

AO represents an oxyalkylene group having 3 or 4 carbon atoms, and examples of the oxyalkylene group include an oxypropylene group, an oxybutylene group, an oxyisobutylene group, an oxytrimethylene group, and an oxytetramethylene group, and an oxypropylene group or an oxybutylene group is preferable.

EO represents an oxyethylene group.

a and b represent average numbers of moles of added AO and EO, respectively, and satisfy 1≤a≤70 and preferably 2≤a≤70, and 1≤b≤70 and preferably 2≤b≤70. a+b is preferably 8 to 100.

M_{AO} representing the molecular weight of AO and M_{EO} representing the molecular weight of EO satisfy

[M_{EO}×b/(M_{AO}×a+M_{EO}×b)]=0.2 to 0.8.

The order in which AO and EO are added is not particularly limited. AO and EO may be added in a block form or a random form, and a random form is preferable. The block form may include not only a two-stage block but also a three or more-stage block.

Specific examples of the alkylene oxide derivative include polyoxyethylene (10 mol) polyoxypropylene (10 mol) dimethyl ether, polyoxyethylene (9 mol) polyoxypropylene (2 mol) dimethyl ether, polyoxyethylene (14 mol) polyoxypropylene (7 mol) dimethyl ether, polyoxyethylene (17 mol) polyoxypropylene (4 mol) dimethyl ether, polyoxyethylene (6 mol) polyoxypropylene (14 mol) dimethyl ether, polyoxyethylene (15 mol) polyoxypropylene (5 mol) dimethyl ether, polyoxyethylene (25 mol) polyoxypropylene (25 mol) dimethyl ether, polyoxyethylene (9 mol) polyoxybutylene (2 mol) dimethyl ether, polyoxyethylene (14 mol) polyoxybutylene (7 mol) dimethyl ether, polyoxyethylene (10 mol) polyoxypropylene (10 mol) diethyl ether, polyoxyethylene (10 mol) polyoxypropylene (10 mol) dipropyl ether, polyoxyethylene (10 mol) polyoxypropylene (10 mol) dibutyl ether, and polyoxyethylene (36 mol) polyoxypropylene (41 mol) dimethyl ether, and among them, polyoxyethylene (9 mol) polyoxypropylene (2 mol) dimethyl ether, polyoxyethylene (14 mol) polyoxypropylene (7 mol) dimethyl ether, or polyoxyethylene (36 mol) polyoxypropylene (41 mol) dimethyl ether is preferable, and polyoxyethylene (14 mol) polyoxypropylene (7 mol) dimethyl ether is particularly preferable.

The alkylene oxide derivative of the present invention can be produced with a known method. For example, the alkylene oxide derivative can be obtained by addition-polymerizing ethylene oxide and an alkylene oxide having 3 to 4 carbon atoms to a compound having a hydroxy group, and then etherifying an alkyl halide in the presence of an alkali catalyst.

One or more kinds of the component (E) can be blended. The amount of the component (E) blended is preferably 0.01 to 10 mass %, and more preferably 0.05 to 5 mass % with respect to the total amount of the cosmetic.

### (F) Nicotinic Acid or its Derivative

The cosmetic according to the present invention can further include (F) nicotinic acid or its derivative. Examples of the derivative of nicotinic acid include nicotinic acid amide, benzyl nicotinate, tocopherol nicotinate, and nicotinic acid β-butoxy ester, and among these, nicotinic acid amide is particularly preferable. Here, nicotinic acid amide is also referred to as nicotinamide or niacinamide, is a derivative of nicotinic acid, and is an amide compound of nicotinic acid (vitamin B3/niacin). Nicotinic acid amide is a water-soluble vitamin and a substance known as one of vitamin B complex, and may be extracted from a natural product (rice bran or the like) or can be synthesized with a known method. For example, those listed in the Japanese Pharmacopoeia Eighteenth Edition can be used.

One or more kinds of the component (F) can be blended. The amount of the component (F) blended is preferably 0.1 to 10 mass %, and more preferably 0.5 to 8 mass % with respect to the total amount of the cosmetic.

### (G) Nonionic Surfactant

The cosmetic according to the present invention can further include (G) a nonionic surfactant. The component (G) is not particularly limited as long as it is a nonionic surfactant usually used in external preparations such as cosmetics and quasi-drugs, and examples of the component (G) include polyoxyalkylene glyceryl fatty acid esters such as PEG-60 glyceryl isostearate, polyoxyethylene hydrogenated castor oils such as PEG-40 hydrogenated castor oil, PEG-60 hydrogenated castor oil, and PEG-100 hydrogenated castor oil, polyoxyethylene-methylpolysiloxane copolymers such as PEG-3 dimethicone, PEG-9 dimethicone, PEG-10 dimethicone, PEG-12 dimethicone, and PEG-9 polydimethylsiloxyethyl dimethicone, polyglycerin fatty acid esters such as polyglyceryl-2 diisostearate and polyglyceryl-2 triisostearate, monoglycerin fatty acid esters such as glyceryl stearate and glyceryl oleate, polyoxyethylene sorbitan fatty acid esters such as polyoxyethylene sorbitan monooleate and polyoxyethylene sorbitan monostearate, sorbitan fatty acid esters such as sorbitan sesquioleate, sorbitan sesquiisostearate, sorbitan isostearate, and sorbitan palmitate.

One or more kinds of the component (G) can be blended. The amount of the component (G) blended is preferably 1.5 mass % or less, and more preferably 0.1 to 1.0 mass % with respect to the total amount of the cosmetic.

In the cosmetic of the present invention, an optional component usually used in cosmetics and pharmaceuticals can be blended in addition to the above-described components. Examples of the optional component include the following components, and one or more kinds of them can be blended as long as an effect of the present invention is exhibited.

Examples of the moisturizer include polyethylene glycol, propylene glycol, dipropylene glycol (DPG), glycerin, 1,3-butylene glycol (BG), erythritol, xylitol, sorbitol, maltitol, chondroitin sulfate, hyaluronic acid, mucoitinsulfuric acid, charonin acid, atelocollagen, cholesteryl-12-hydroxystearate, sodium lactate, bile acid salts, dl-pyrrolidone carboxylates, short-chain soluble collagen, a chestnut rose extract, a yarrow extract, and a melilot extract.

Examples of the lower alcohol include ethanol, 1-propanol, 2-propanol, isobutyl alcohol, and t-butyl alcohol.

Examples of the thickener include gum arabic, carrageenan, karaya gum, tragacanth gum, carob gum, quince seed (Pyrus cydonia), casein, dextrin, gelatin, sodium pectinate, sodium alginate, methylcellulose, ethylcellulose, CMC, hydroxyethylcellulose, hydroxypropylcellulose, PVA, PVM, PVP, sodium polyacrylate, a carboxyvinyl polymer, locust bean gum, guar gum, tamarind gum, dialkyldimethylammonium sulfate cellulose, xanthan gum, magnesium aluminum silicate, bentonite, hectorite, magnesium aluminum silicate (VEEGUM), laponite, and silicic anhydride.

Examples of the sequestering agent include 1-hydroxyethane-1,1-diphosphonic acid, 1-hydroxyethane-1,1-diphosphonic acid tetrasodium salt, edetate disodium, edetate trisodium, edetate tetrasodium, sodium citrate, sodium polyphosphate, sodium metaphosphate, gluconic acid, phosphoric acid, citric acid, succinic acid, edetic acid, and trisodium hydroxyethyl ethylenediamine triacetate.

Examples of the neutralizer include 2-amino-2-methyl-1-propanol, 2-amino-2-methyl-1,3-propanediol, potassium hydroxide, sodium hydroxide, triethanolamine, and sodium carbonate.

Examples of a pH adjuster include buffers such as lactic acid-sodium lactate, citric acid-sodium citrate, and succinic acid-sodium succinate.

Examples of the antioxidant include dibutylhydroxytoluene, butylhydroxyanisole, sodium pyrosulfite, and gallic acid esters.

Examples of the preservative include paraoxybenzoic acid esters such as methylparaben, ethylparaben, and butylparaben, benzoic acid, salicylic acid, sorbic acid, parachlorometacresol, hexachlorophene, benzalkonium chloride, chlorhexidine chloride, trichlorocarbanilide, photosensitizers, and phenoxyethanol.

Examples of the drug include ascorbic acid (vitamin C), tranexamic acid, kojic acid, ellagic acid, arbutin, alkoxysalicylic acid, glycyrrhizic acid, tocopherol, retinol, salts and derivatives thereof (for example, sodium L-ascorbate, L-ascorbic acid ester magnesium salt, L-ascorbic acid glucoside, 2-O-ethyl-L-ascorbic acid, 3-O-ethyl-L-ascorbic acid, 4-methoxysalicylic acid sodium salt, 4-methoxysalicylic acid potassium salt, dipotassium glycyrrhizinate, stearyl glycyrrhizinate, tocopherol acetate, retinol acetate, and retinol palmitate), caffeine, tannin, verapamil and its derivatives, a licorice extract, glabridin, a hot water extract of Pyracantha fortuneana seed, various herbal medicines, hydrolyzed silk, hydrolyzed conchiolin, a tea extract, a Potentilla erecta root extract, an Angelica keiskei leaf/stem extract, an Aloe barbadensis leaf extract, a cherry leaf extract, an Angelica acutiloba Kitagawa root extract, a Citrus depressa peel extract, an Iris florentina root extract, an Eucheuma serra/Grateloupia sparsa/Saccharina angustata/Ulva linza/Undaria pinnatifida extract, a Typha angustifolia spike extract, an Isodonis japonicus leaf/stalk extract, a Camellia japonica seed extract, a Saccharina angustata/Undaria pinnatifida extract, a Bupleurum falcatum root extract, a Nasturtium officinale leaf/stem extract, a Cinnamomum cassia bark extract, a rosemary leaf oil, a lavender oil, glutamic acid, trimethylglycine, chlorphenesin, and menthoxypropanediol.

In addition, an ultraviolet absorber, a powder component, an aroma chemical, and the like can also be appropriately blended.

Examples of the cosmetic according to the present invention include skin care cosmetics (such as lotions, milky lotions, creams, beauty essences, packs, and masks), makeup cosmetics (such as foundations and makeup bases), skin cleaning agents (such as face washes and makeup removers), sunscreen cosmetics, and ointments. These embodiments are merely examples, and the cosmetic according to the present invention is not limited to these embodiments.

The method of emulsification for the cosmetic of the present invention is not particularly limited, and the cosmetic can be produced in accordance with a conventional method.

The average emulsified particle size is not particularly limited, but is preferably 0.1 to 30 μm, and more preferably 0.5 to 20 μm. The average emulsified particle size can be measured using a microscope.

The viscosity is not particularly limited, but is preferably 100 to 100,000 mPa·s, more preferably 500 to 30,000 mPa·s, and particularly preferably 1,000 to 15,000 mPa·s. The viscosity can be measured at 30° C. using a B type viscometer.

### EXAMPLES

The present invention will be described specifically with reference to the following Examples, but the present invention is not limited to these Examples. The content is shown in mass % with respect to the total amount unless otherwise specified.

### Examples 1 to 13 and Comparative Examples 1 and 2

Cosmetics of Examples 1 to 13 and Comparative Examples 1 and 2 were prepared at combinations shown in Table 1. The numerical values in the table are shown in mass %.

### [Table 1]

TABLE 1

| | | | Example | | | | | | | |
|---|---|---|---|---|---|---|---|---|---|---|
| | | | 1 | 2 | 3 | 4 | 5 | 6 | 7 | 8 |
| Composition | (A) | 1-(2-Hydroxyethyl)-2- | 1.5 | 0.1 | 3 | 1.5 | 1.5 | 1.5 | 1.5 | 1.5 |
| | | imidazolidinone | | | | | | | | |
| | (B) | Acrylates/C10-30 alkyl | 0.05 | 0.05 | 0.05 | 0.05 | 0.05 | 0.05 | 0.05 | 0.05 |
| | | acrylate crosspolymer | | | | | | | | |
| | (C) | Cetyl ethylhexanoate | 1 | 1 | 1 | 1 | 1 | 1 | 1 | 1 |
| | | Mineral oil | 1.5 | 1.5 | 1.5 | 1.5 | 1.5 | 1.5 | 1.5 | 1.5 |
| | | Dimethicone | 1 | 1 | 1 | 1 | 1 | 1 | 1 | 1 |
| | | Diphenylsiloxy phenyl | 0.5 | 0.5 | 0.5 | 0.5 | 0.5 | 0.5 | 0.5 | 0.5 |
| | | trimethicone | | | | | | | | |
| | (D) | Water | Balance | Balance | Balance | Balance | Balance | Balance | Balance | Balance |
| | (E) | PEG/PPG-14/7 | — | — | — | 1 | 0.1 | — | — | — |
| | | dimethyl ether | | | | | | | | |
| | (F) | Nicotinic acid amide | — | — | — | — | — | 5 | 1 | — |
| | (G) | Glyceryl stearate | — | — | — | — | — | — | — | 0.25 |
| | | PEG-60 glyceryl | — | — | — | — | — | — | — | 0.15 |
| | | isostearate | | | | | | | | |

| | | | | | | | | | |
|---|---|---|---|---|---|---|---|---|---|
| | Ethanol | 5 | 5 | 5 | 5 | 5 | 5 | 5 | 5 |
| | Glycerin | 8 | 8 | 8 | 8 | 8 | 8 | 8 | 8 |
| | DPG | 9 | 9 | 9 | 9 | 9 | 9 | 9 | 9 |
| | Xanthan gum | 0.05 | 0.05 | 0.05 | 0.05 | 0.05 | 0.05 | 0.05 | 0.05 |
| | Carbomer | 0.22 | 0.22 | 0.22 | 0.22 | 0.22 | 0.22 | 0.22 | 0.22 |
| | Potassium hydroxide | 0.09 | 0.09 | 0.09 | 0.09 | 0.09 | 0.09 | 0.09 | 0.09 |
| | EDTA-2Na | 0.02 | 0.02 | 0.02 | 0.02 | 0.02 | 0.02 | 0.02 | 0.02 |
| | Phenoxyethanol | 0.5 | 0.5 | 0.5 | 0.5 | 0.5 | 0.5 | 0.5 | 0.5 |
| | Total | 100 | 100 | 100 | 100 | 100 | 100 | 100 | 100 |
| | Freshness | A | A | A | A | A | A | A | A |
| Evaluation | Moist feel | A | B | A | AA | A | AA | AA | AA |
| | Absence of sticky feel | A | A | B | AA | AA | A | A | A |

| | | | | | | | | Comparative | |
|---|---|---|---|---|---|---|---|---|---|
| | | | Example | | | | | Example | |
| | | | 9 | 10 | 11 | 12 | 13 | 1 | 2 |
| Composition | (A) | 1-(2-Hydroxyethyl)-2- | 1.5 | 1.5 | 1.5 | 1.5 | 1.5 | — | 1.5 |
| | | imidazolidinone | | | | | | | |
| | (B) | Acrylates/C10-30 alkyl | 0.05 | 0.05 | 0.05 | 0.1 | 0.1 | 0.05 | — |
| | | acrylate crosspolymer | | | | | | | |
| | (C) | Cetyl ethylhexanoate | 2 | 2 | 1 | 3 | 15 | 1 | 1 |
| | | Mineral oil | 3 | 3 | 1.5 | 4.5 | 7 | 1.5 | 1.5 |
| | | Dimethicone | 3 | 3 | 1 | 3 | 2.5 | 1 | 1 |
| | | Diphenylsiloxy phenyl | 0.5 | 0.5 | 0.5 | 1.5 | 0.5 | 0.5 | 0.5 |
| | | trimethicone | | | | | | | |
| | (D) | Water | Balance | Balance | Balance | Balance | Balance | Balance | Balance |
| | (E) | PEG/PPG-14/7 | — | — | 1 | — | — | — | — |
| | | dimethyl ether | | | | | | | |
| | (F) | Nicotinic acid amide | — | — | 5 | — | — | — | — |
| | (G) | Glyceryl stearate | 0.6 | 0.75 | 0.25 | 0.25 | 0.25 | — | 0.25 |
| | | PEG-60 glyceryl | 0.36 | 0.45 | 0.15 | 0.15 | 0.15 | — | 0.15 |
| | | isostearate | | | | | | | |

| | | | | | | | | |
|---|---|---|---|---|---|---|---|---|
| | Ethanol | 5 | 5 | 5 | 5 | 5 | 5 | 5 |
| | Glycerin | 8 | 8 | 8 | 8 | 8 | 8 | 8 |
| | DPG | 9 | 9 | 9 | 9 | 9 | 9 | 9 |
| | Xanthan gum | 0.05 | 0.05 | 0.05 | 0.05 | 0.05 | 0.05 | 0.05 |
| | Carbomer | 0.22 | 0.22 | 0.22 | 0.22 | 0.22 | 0.22 | 0.22 |
| | Potassium hydroxide | 0.09 | 0.09 | 0.09 | 0.11 | 0.11 | 0.09 | 0.08 |
| | EDTA-2Na | 0.02 | 0.02 | 0.02 | 0.02 | 0.02 | 0.02 | 0.02 |
| | Phenoxyethanol | 0.5 | 0.5 | 0.5 | 0.5 | 0.5 | 0.5 | 0.5 |
| | Total | 100 | 100 | 100 | 100 | 100 | 100 | 100 |
| | Freshness | A | A | A | A | A | B | C |
| Evaluation | Moist feel | AA | AA | AA | AA | AA | D | B |
| | Absence of sticky feel | B | C | AA | B | C | C | D |

The cosmetic prepared above was applied to the skin of 10 expert panelists, and the panelists evaluated “freshness” during the application, a “moist feel” after the application, and “absence of a sticky feel” after the application. Determination was made in accordance with the following criteria on the basis of the evaluation of each expert panelist. The obtained results are as shown in Table 1.

### [Freshness]

AA: All of the 10 panelists answered that a fresh feel was present.

A: Seven or more and 9 or less of the 10 panelists answered that a fresh feel was present.

B: Four or more and 6 or less of the 10 panelists answered that a fresh feel was present.

C: One or more and 3 or less of the 10 panelists answered that a fresh feel was present.

D: All of the 10 panelists answered that a fresh feel was absent.

### [Moist Feel]

AA: All of the 10 panelists answered that a moist feel of the skin was present.

A: Seven or more and 9 or less of the 10 panelists answered that a moist feel of the skin was present.

B: Four or more and 6 or less of the 10 panelists answered that a moist feel of the skin was present.

C: One or more and 3 or less of the 10 panelists answered that a moist feel of the skin was present.

D: All of the 10 panelists answered that a moist feel of the skin was absent.

### [Absence of Sticky Feel]

AA: All of the 10 panelists answered that a sticky feel was absent.

A: Seven or more and 9 or less of the 10 panelists answered that a sticky feel was absent.

B: Four or more and 6 or less of the 10 panelists answered that a sticky feel was absent.

C: One or more and 3 or less of the 10 panelists answered that a sticky feel was absent.

D: All of the 10 panelists answered that a sticky feel was present.

### Formulation Examples 1 and 2

Formulation Examples of the cosmetic of the present invention are shown in the following Tables 2 and 3. The numerical values in Tables 2 and 3 are shown in mass %.

### [Table 2]

TABLE 2

| | Formulation Example 1 |
|---|---|
| Component | (milky lotion) |
| 1-(2-Hydroxyethyl)-2-imidazolidinone | 1.5 |
| Acrylates/C10-30 alkyl acrylate crosspolymer | 0.05 |
| Mineral oil | 1.5 |
| Dimethicone | 1 |
| Cetyl ethylhexanoate | 1 |
| Phytosteryl macadamiate | 0.01 |
| Diphenylsiloxy phenyl trimethicone | 0.5 |
| Water | Balance |
| PEG/PPG-17/4 dimethyl ether | 0.1 |
| PEG/PPG-14/7 dimethyl ether | 0.05 |
| Nicotinic acid amide | 5 |
| Glyceryl stearate | 0.25 |
| PEG-60 glyceryl isostearate | 0.15 |
| Ethanol | 5 |
| Glycerin | 8 |
| BG | 0.03 |
| DPG | 9 |
| Menthoxypropanediol | 0.04 |
| Erythritol | 0.05 |
| Xanthan gum | 0.05 |
| Carbomer | 0.22 |
| Potassium hydroxide | 0.08 |
| Dipotassium glycyrrhizinate | 0.05 |
| Rosemary leaf oil | 0.02 |
| Lavender oil | 0.01 |
| Glutamic acid | 0.01 |
| Eucalyptus oil | 0.01 |
| Tea extract | 0.01 |
| Potentilla erecta root extract | 0.01 |
| Angelica keiskei leaf/stem extract | 0.01 |
| Aloe barbadensis leaf extract | 0.01 |
| EDTA-2Na | 0.02 |
| Sodium pyrosulfite | 0.01 |
| Tocopherol | 0.01 |
| Phenoxyethanol | 0.5 |
| Aroma chemical | 0.06 |

### [Table 3]

TABLE 3

| | Formulation Example |
|---|---|
| Component | 2 (cream) |
| (2-Hydroxyethyl)-2-imidazolidinone | 1.5 |
| Acrylates/C10-30 alkyl acrylate crosspolymer | 0.02 |
| Cetyl ethylhexanoate | 6 |
| Hydrogenated polydecene | 6 |
| Dimethicone | 5 |
| Squalane | 3 |
| Stearyl alcohol | 2 |
| Glyceryl stearate | 2 |
| Shea butter | 2 |
| Behenyl alcohol | 1 |
| Isostearic acid | 0.5 |
| Hydrogenated palm oil | 0.5 |
| Palm kernel oil | 0.3 |
| Palm oil | 0.2 |
| Water | Balance |
| PEG/PPG-17/4 dimethyl ether | 0.1 |
| PEG/PPG-14/7 dimethyl ether | 0.05 |
| Nicotinic acid amide | 5 |
| PEG-60 glyceryl isostearate | 2 |
| Ethanol | 0.04 |
| Glycerin | 3 |
| BG | 6 |
| Xylitol | 5 |
| Xanthan gum | 0.1 |
| Sodium polyacrylate | 0.01 |
| Potassium hydroxide | 0.02 |
| 2-O-ethyl ascorbic acid | 0.05 |
| Cherry leaf extract | 0.01 |
| Angelica acutiloba Kitagawa root extract | 0.01 |
| Citrus depressa peel extract | 0.01 |
| Iris florentina root extract | 0.01 |
| Eucheuma serra/Grateloupia sparsa/ | 0.01 |
| Saccharina angustata/Ulva | |
| Typha angustifolia spike extract | 0.01 |
| Isodonis japonicus leaf/stalk extract | 0.01 |
| Camellia japonica seed extract | 0.01 |
| Saccharina angustata/Undaria pinnatifida extract | 0.01 |
| Tea extract | 0.01 |
| Hydrolyzed silk | 0.01 |
| Bupleurum falcatum root extract | 0.01 |
| Nasturtium officinale leaf/stem extract | 0.01 |
| Hydrolyzed conchiolin | 0.01 |
| Cinnamomum cassia bark extract | 0.01 |
| EDTA-2Na | 0.03 |
| Sodium pyrosulfite | 0.02 |
| Sodium metaphosphate | 0.01 |
| Tocopherol | 0.01 |
| Citric acid | 0.01 |
| Phenoxyethanol | 0.5 |
| Chlorphenesin | 0.2 |
| Silica | 3 |
| Mica | 0.5 |
| Titanium oxide | 0.5 |
| Iron oxide | 0.01 |
| Aroma chemical | 0.2 |

1. An oil-in-water emulsion cosmetic comprising:
(A) a cyclic carboxamide derivative or a salt of the cyclic carboxamide derivative, the cyclic carboxamide derivative represented by Formula (1): 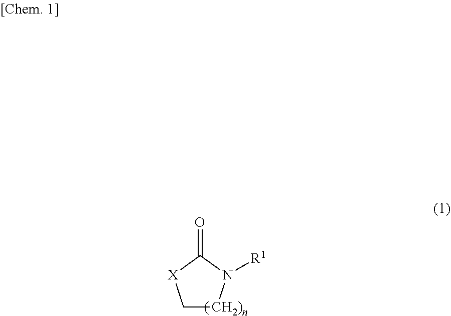
wherein
R¹ represents a hydrocarbon group having 1 to 6 carbon atoms optionally substituted with a hydroxy group or R¹ represents a hydrogen atom,
X represents —CH₂— or —N(R²)— wherein R² represents a hydrocarbon group having 1 to 6 carbon atoms optionally substituted with a hydroxy group or R² represents a hydrogen atom, and
n is an integer of 1 to 3;
(B) an alkyl-modified carboxyvinyl polymer;
(C) an oil; and
(D) water.

2. The oil-in-water emulsion cosmetic according to claim 1,
wherein in Formula (1) of the component (A),
R¹ represents a hydroxyalkyl group having 1 to 3 carbon atoms,
X represents —CH₂— or —NH—, and
n is 1.

3. The oil-in-water emulsion cosmetic according to claim 1, wherein the component (A) is 1-(2-hydroxyethyl)-2-imidazolidinone.

4. The oil-in-water emulsion cosmetic according to claim 1, wherein an amount of the component (A) blended is 0.05 to 5 mass % with respect to a total amount of the oil-in-water emulsion cosmetic.

5. The oil-in-water emulsion cosmetic according to claim 1, wherein the component (B) is an acrylates/C10-30 alkyl acrylate crosspolymer.

6. The oil-in-water emulsion cosmetic according to claim 1, wherein an amount of the component (C) blended is 30 mass % or less with respect to the total amount of the oil-in-water emulsion cosmetic.

7. The oil-in-water emulsion cosmetic according to claim 1, further comprising (E) an alkylene oxide derivative represented by Formula (2):
R³O-[(AO)ₐ(EO)_{b}]—R⁴ (2)
wherein
R³ and R⁴ each independently represent an alkyl group having 1 to 4 carbon atoms,
AO represents an oxyalkylene group having 3 or 4 carbon atoms,
EO represents an oxyethylene group,
a and b represent average numbers of moles of added AO and EO, respectively, and satisfy 1≤a≤70 and 1≤b≤70, and
M_{AO} representing a molecular weight of AO and M_{EO} representing a molecular weight of EO satisfy [M_{EO}×b/(M_{AO}×a+M_{EO}×b)]=0.2 to 0.8.

8. The oil-in-water emulsion cosmetic according to claim 1, further comprising (F) nicotinic acid or a derivative of nicotinic acid.

9. The oil-in-water emulsion cosmetic according to claim 1, further comprising (G) a nonionic surfactant.

10. The oil-in-water emulsion cosmetic according to claim 9, wherein an amount of the component (G) blended is 1.5 mass % or less with respect to the total amount of the oil-in-water emulsion cosmetic.
